SEQUENCE LISTING (6 sequences)

SEQ ID NO: 1 - DNA, 78 bp, Artificial Sequence
  Synthetic oligonucleotide
actgggatccatggccctgtggacacgcctgcggcccctgctggccctgctggcgctctggcccccccccccggcccg

SEQ ID NO: 2 - DNA, 80 bp, Artificial Sequence
  Synthetic oligonucleotide
ctccgcacaccaggtacagcgcctccaccaggtgggagccacacagatgctggttgacgaaggcgcgggccggggggggg

SEQ ID NO: 3 - DNA, 76 bp, Artificial Sequence
  Synthetic oligonucleotide
acctggtgtgcggagagcgcggcttcttctacacgcccaaggctgctaagggcattgtggaacaatgctgtaccag

SEQ ID NO: 4 - DNA, 76 bp, Artificial Sequence
  Synthetic oligonucleotide
gtgtggggctgcctgcaggctgcgtctagttgcagtagttctccagctggtagagggagcagatgctggtacagca

SEQ ID NO: 5 - DNA, 80 bp, Artificial Sequence
  Synthetic oligonucleotide
caggcagccccacacccgccgcctcctgcaccgagagagatggaataaagcccttgaaccagccctgctgtgccgtctgt

SEQ ID NO: 6 - DNA, 99 bp, Artificial Sequence
  Synthetic oligonucleotide
tgacgcggccgcagcgtggagagagctgggaggggctcacaacagtgccgggaagtggggcttggcccagggcccccaagacacacagcaggcacagca

(19) United States
(12) Patent Application Publication
(10) Pub. No.: US 2009/0228999 A1
(43) Pub. Date: Sep. 10, 2009

(54) Process of Making Transgenic Mammals That Produce Exogenous Proteins in Milk and Transgenic Mammals Produced Thereby

(75) Inventors: A. BERCOVICH, Ciudad de Buenos Aires (AR); A. PRYNC, Ciudad de Buenos Aires (AR); N. Fernandez, Ciudad de Buenos Aires (AR); C. Melo, Provincia de Buenos Aires (AR); M. Criscuolo, Ciudad de Buenos Aires (AR)

Correspondence Address: STERNE, KESSLER, GOLDSTEIN & FOX P.L.L.C., 1100 NEW YORK AVENUE, N.W., WASHINGTON, DC, 20005, US

(73) Assignee: Sterrenbeld Biotechnologie North America, Inc., Wilmington, DE (US)

(21) Appl. No.: 12/138,526

(22) Filed: Jun. 13, 2008

Related U.S. Application Data
(60) Provisional application No. 60/929,098, filed on Jun. 13, 2007.
(60) Provisional application No. 60/929,095, filed on Jun. 13, 2007.

(51) Int. Cl. A01K 67/033; C12N 15/85

(52) U.S. Cl. 800/15; 800/13; 800/16; 800/17; 800/14; 435/320.1

(57) ABSTRACT

The invention relates to a non-human transgenic mammal that is useful for the production of a protein of interest that may be toxic to the mammal. The mammal is characterized by the fact that it is transgenic for the production in its milk of an inactive form of the protein of interest, preferably recombinant human insulin. It is not possible to produce recombinant human insulin in transgenic mammals since this molecule has a certain degree of biological activity in the mammals and could be toxic to the mammal. Thus, the invention involves cloning a genetic construct comprising a sequence encoding a modified human insulin precursor under the control of a beta casein promoter in an expression vector. It also involves transfecting the expression plasmid into fetal bovine somatic cells, such as fibroblasts, and enucleating bovine oocytes by nuclear transfer to generate transgenic embryos. The invention gives rise to transgenic bovine that will be able to produce a modified human insulin precursor in their mammary glands. Afterwards, the milk of these transgenic mammals can be collected, the modified human insulin precursor can be converted in vitro into recombinant human insulin, and the recombinant human insulin can be purified to homogeneity as a pure biopharmaceutical product.

### BACKGROUND OF THE INVENTION

### Field of the Invention

Protein factors and hormones involved in human health care have been currently produced by the pharmaceutical industry by extraction or by recombinant technology in past decades. Expression of genetic constructs involving the desired genes were successfully accomplished in bacteria, yeast or mammalian cell lines. However, the use of mammalian cell cultures to obtain complex proteins, such as those which require a proper glycosylation pattern, involves high cost procedures.

Recombinant DNA technology has been used increasingly over the past decade for the production of commercially important biological materials. To this end, the DNA sequences encoding a variety of medically important human proteins have been cloned. These include insulin, plasminogen activator, alpha1-antitrypsin and coagulation factors VIII and IX. At present, even with the emergent recombinant DNA techniques, these proteins are usually purified from blood and tissue, an expensive and time consuming process which may carry the risk of transmitting infectious agents such as those causing AIDS and hepatitis.

Although the expression of DNA sequences in bacteria to produce the desired medically important protein looks like an attractive proposition, in practice the bacteria often prove unsatisfactory as hosts because in the bacterial cell foreign proteins are unstable and are not processed correctly.

Recognizing this problem, the expression of cloned genes in mammalian tissue culture has been attempted and has in some instances proved a viable strategy. However, batch fermentation of mammalian cells is an expensive and technically demanding process.

There is therefore a need for a high yield, low cost process for the production of biological substances such as correctly modified eukaryotic polypeptides. The absence of agents that are infectious to humans would be an advantage in such a process.

The possibility of obtaining transgenic mammals, like cattle, for a desired gene, with the aim of getting large amounts of a human protein in milk, has been of great interest to the industry. Several groups in the literature report their success on producing human serum albumin, alpha anti-trypsin, and some other examples in transgenic cows or goats.

Many experiments have been previously performed in mice or rats, and transgene expression was always preferred to be confined to the mammary glands since beta casein or lactalbumin promoters were employed, which respond only to mammary gland transcription factors in lactating females.

The expression of a heterologous protein exclusively in milk is meant to avoid undesired influence on the health of the host mammal and provide an easy method for purification.

Expression of heterologous proteins in bacteria or cell culture may be prevented or impeded due to a toxic effect of the recombinant protein on the host mammal. Many examples can be found in the literature where a certain protein, even a naturally non-toxic one, cannot be expressed in a particular system because it is harmful to the host, even causing its death. The cause of death may be the high concentration of the protein inside the cell, the high concentration of secreted protein or a specific interaction with the protein and some cellular component that causes cytopathic activity in the foreign host.

Several methods have been developed to overcome these drawbacks to achieve heterologous gene expression of toxic proteins, including using fusion proteins, modifying the original protein sequence, separately expressing the different polypeptides of a protein, etc. (See Protein Expression and Purification, 2001 August; 22(3):422-9).

A similar effect may result when expressing recombinant proteins in transgenic cattle. In the generation of a transgenic mammal, a cell is transfected to obtain a transgenic clone carrying the heterologous gene of interest and is then used to generate the transgenic mammal. This process generally leads to the insertion of the sequence of interest in the host genome, an event that in turn should lead to the expression of the heterologous protein in the target tissue or gland if a specific promoter was used, or systemically if a general promoter was employed. The level of protein expression will depend on a variety of factors, including the location within the genome where the insertion took place.

Even when the gene expression is directed by a tissue specific promoter, leakage of the foreign protein into the peripheral circulation system has been observed in many different mammalian species with several proteins (See Life Sciences, 2006 Jan. 25; 78(9):1003-9. Epub 2005 Sep. 15; and Journal of Biotechnology, 2006 Jul. 13; 124(2):469-72. Epub 2006 May 23). This leakage may have relevant biological consequences depending on the level of expression, level of leakage, nature of the heterologous protein, relation between species (host and foreign protein) and the ability of the heterologous protein to interact with host receptors. Given the similarity to the host homologous protein, some of these transgenically expressed proteins may exert their natural biological activity on the foreign host and may cause a pathological effect that could cause the death of the mammal (See Endocrinology 1997 July; 138(7):2849-55). In addition, it is possible that the heterologous protein does not affect the mammal's health through an interaction with the corresponding homologous host receptors, but through a toxic, non-specific effect that occurs when some heterologous proteins are expressed in bacteria.

This invention provides innovative solutions to the drawbacks currently associated with expressing a protein in transgenic mammals that has a toxic effect on the mammals.

### SUMMARY OF THE INVENTION

The invention relates to a non-human mammal which is useful for the production of a protein of interest that may be toxic to the mammal. This mammal is characterized by the fact that it is transgenic for the production in its milk of an inactive form of the protein of interest. The inactive form of the protein of interest is a form of the protein of interest that is not toxic to the non-human transgenic mammal that expresses the protein of interest. As used herein, toxic means causing serious harm or death. An inactive form of the protein of interest may have some biological activity in the non-human transgenic mammal that expresses the inactive form of the protein of interest; however, the inactive form of the protein of interest is not toxic to the non-human transgenic mammal (i.e., the mammal does not die and does not suffer serious harm). The inactive form of the protein can be activated in vitro. This inactive protein, possibly a non-natural species of the protein, may be, but is not limited to, a recombinant modified human insulin precursor. The protein of interest may be, but is not limited to, recombinant human insulin. The non-human transgenic mammal may be, but is not limited to, a mammal of bovine species.

The invention further relates to a plasmid that provides for the expression of the inactive form of the protein of interest in the mammary cells of mammals in which the expression is regulated by the beta casein promoter.

The present invention further relates to a method of production, employing non-human transgenic mammals, of a protein of interest that may be toxic to the non-human transgenic mammals. The potential toxicity of the protein is avoided by expressing the protein as an inactive protein. This inactive protein, possibly a non-natural species of the protein, may be, but is not limited to, a recombinant modified human insulin precursor. The protein of interest may be, but is not limited to, recombinant human insulin. The non-human transgenic mammal may be, but is not limited to, a mammal of bovine species.

The invention also relates to a method of producing recombinant insulin, comprising making a non-human transgenic mammal that produces a recombinant modified insulin precursor in its milk, obtaining the milk from the non-human transgenic mammal, purifying the precursor from the milk, subjecting the purified precursor to enzymatic cleavage and transpeptidation in order to yield recombinant insulin, and purifying the recombinant insulin. The recombinant insulin may be, but is not limited to, recombinant human insulin. The transgenic mammal may be, but is not limited to, a mammal of bovine species.

### BRIEF DESCRIPTION OF THE DRAWINGS/FIGURES

FIG. 1 shows a scheme of expression plasmid pβmhuIP, containing the genetic sequence which encodes the modified human insulin precursor (mhuIP) and a promoter that directs its expression to mammary cells.

FIG. 2 shows a scheme Start Construction, comprising the sequence encoding mhuIP.

FIG. 3 shows a scheme of expression plasmid pNJK IP, containing the genetic sequence which encodes the modified human insulin precursor (mhuIP), a promoter that directs its expression to mammary cells, and a fragment of the coding sequence of the chicken β globin insulator.

FIG. 4 shows a scheme of expression plasmid pβKLE IP, containing the genetic sequence which encodes the modified human insulin precursor (mhuIP), a promoter that directs its expression to mammary cells, a large portion of the coding sequence of the bovine alfa lactalbumin gene, and an enterokinase cleavage site.

### DETAILED DESCRIPTION OF THE INVENTION

The invention relates to a non-human mammal which is useful for the production of a protein of interest that may be toxic to the mammal. That mammal is characterized by the fact that it is transgenic for the production of an inactive form of the protein of interest in its milk. The term inactive protein refers to a form of the protein of interest that is not toxic to the non-human transgenic mammal that expresses the protein of interest. In a further embodiment of the invention, the term inactive protein refers to a protein that lacks biological activity without further post-translational modification. Examples of inactive proteins include precursor proteins (i.e., propeptides), proteins that contain modifications (i.e., amino acid substitutions, additions or deletions when compared to the native protein) that render the protein biologically inactive without further processing, or modified precursor proteins (i.e., propeptides that contain amino acid substitutions, additions or deletions when compared to the native propeptide). In other words, the potential toxicity of the protein of interest is avoided by expressing the protein as an inactive protein that does not kill a non-human transgenic mammal that expresses the protein of interest. This inactive protein, possibly a non-natural species of the protein, may be, but is not limited to, precursors, modified precursors or modified forms of the following: antibodies, hormones, growth factors, enzymes, clotting factors, apolipoproteins, receptors, drugs, pharmaceuticals, bioceuticals, nutraceuticals, oncogenes, tumor antigens, tumor suppressors, cytokines, viral antigens, parasitic antigens, and bacterial antigens. Preferably, the inactive protein is a recombinant modified insulin precursor that does not cause hypoglycemia in a non-human transgenic mammal that expresses the modified insulin precursor. More preferably, the inactive protein is a recombinant modified mammalian insulin precursor, and most preferably, a recombinant modified human insulin precursor. The protein of interest may be, but is not limited to, a recombinant insulin, more preferably, a recombinant mammalian insulin, and, most preferably, recombinant human insulin. This non-human mammal may be, but is not limited to, a mammal of bovine species. Other species of transgenic mammals may be, but are not limited to, porcine species, ovine species, caprine species, or rodent species.

Insulin is the primary hormone responsible for controlling the transport, utilization and storage of glucose in the body. The β-cells of the pancreas secrete a single chain precursor of insulin, known as proinsulin. Proinsulin is made up of three domains: an amino-terminal B chain, a carboxyl-terminal A chain, and a connecting peptide in the middle known as the C peptide. Proteolysis of proinsulin results in removal of certain basic amino acids in the proinsulin chain along with the connecting peptide (C peptide) to yield the biologically active polypeptide insulin.

In embodiments, a modified protein is a form of the protein that is not the naturally occurring form of the protein. In embodiments, the modified insulin precursor contains an amino-terminal B chain and a carboxyl-terminal A chain. However, the modified insulin precursor contains a modified C peptide. In the modified insulin precursor, the amino acids encoding the connecting C peptide that is found in naturally occurring proinsulin is replaced by amino acids that are not found in naturally occurring proinsulin. In embodiments, the modified C peptide contains the following three amino acids: Ala-Ala-Lys. Furthermore, the modified insulin precursor may contain a modified B chain. In embodiments, the modified B chain contains all but the C-terminal amino acid of the naturally occurring B chain.

In further embodiments, the modified insulin precursor is a modified human insulin precursor consisting of 53 amino acids, with a molecular weight of about 6 kD. The modified human insulin precursor contains a modified B chain that has amino acids 1-29 of the naturally occurring B chain, and a modified C peptide with three amino acids, Ala-Ala-Lys. The modified human insulin precursor may be subjected to enzymatic cleavage and transpeptidation in order to yield human insulin, which is essential for the treatment of diabetes and its applications are well established.

The invention also relates to a non-human mammal which is transgenic for the production of a recombinant modified human insulin precursor in its milk, characterized by the fact that the recombinant modified human insulin precursor does not render the mammal non-viable.

The invention further relates to a transgenic mammal of bovine species that is useful for the production of recombinant human insulin. Human insulin is known to be active in cattle. Cattle that express human insulin in its mature form might be expected to exhibit symptoms associated with hypoglycemia since transgenic protein can leak into the bloodstream. Therefore, transgenic cattle that express recombinant human insulin may be non-viable. However, the present invention overcomes this limitation and allows for expression in a transgenic mammal of a protein that may be toxic to the transgenic mammals. The present invention expresses an inactive form of a protein of interest. The inactive protein is purified from the milk of the transgenic mammal, and converted in vitro into the mature (i.e., active) form of the protein. Undoubtedly, a mammal, such as a cow, which is useful as a means of producing a therapeutic protein (e.g., human insulin) that when expressed is harmful to the mammal constitutes an unexpected and innovative contribution.

The invention further relates to a non-human transgenic mammal that produces a recombinant modified insulin precursor in its milk, whose genome comprises an integrated plasmid, the plasmid comprising a nucleic acid sequence encoding a modified insulin precursor operably linked to a promoter that directs the expression of the sequence in mammary cells of the mammal. The non-human mammal may be, but is not limited to, a mammal of bovine species. Other species of transgenic mammals may be, but are not limited to, porcine species, ovine species, caprine species, or rodent species. The modified insulin precursor may be a modified mammalian insulin precursor, more preferably, a modified human insulin precursor, a modified bovine insulin precursor, a modified porcine insulin precursor, a modified ovine insulin precursor, a modified caprine insulin precursor, a modified rodent insulin precursor, and most preferably a modified human insulin precursor. The promoter may be a beta casein promoter. Suitable beta casein promoters include, but are not limited to, a bovine beta casein promoter or a caprine beta casein promoter. Other beta casein promoters include, but are not limited to, a porcine beta casein promoter, an ovine beta casein promoter, or a rodent beta casein promoter. The integrated plasmid may also contain an antibiotic resistance gene such as the neomycin resistance gene. Further, the integrated plasmid may be pβmhuIP. The integrated plasmid can also be pNJK IP or pβKLE IP.

The invention further relates to a non-human transgenic mammal in which the above described integrated plasmid is found in both the somatic cells and the germ cells of the mammal.

The invention further relates to a non-human transgenic mammal of bovine species that produces a recombinant modified human insulin precursor in its milk, whose genome comprises an integrated plasmid, the plasmid comprising a nucleic acid sequence encoding the modified human insulin precursor and a beta casein promoter that directs expression of the sequence in mammary cells of the mammal. Suitable beta casein promoters include, but are not limited to, a bovine beta casein promoter or a caprine beta casein promoter. Other beta casein promoters may be, but are not limited to, a porcine beta casein promoter, an ovine beta casein promoter, or a rodent beta casein promoter. The integrated plasmid may contain an antibiotic resistance gene such as the neomycin resistance gene. Further, the integrated plasmid may be pβmhuIP. The integrated plasmid can also be pNJK IP or pβKLE IP.

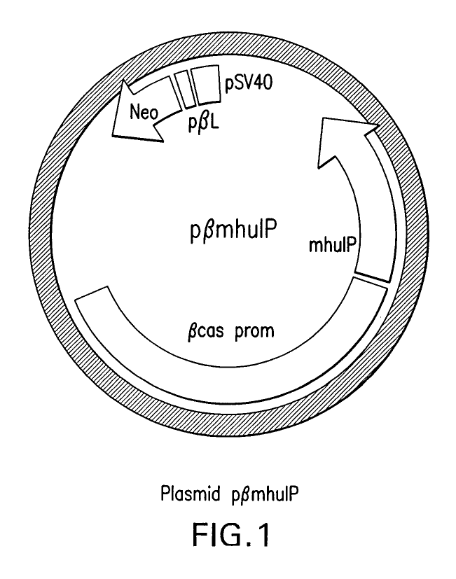

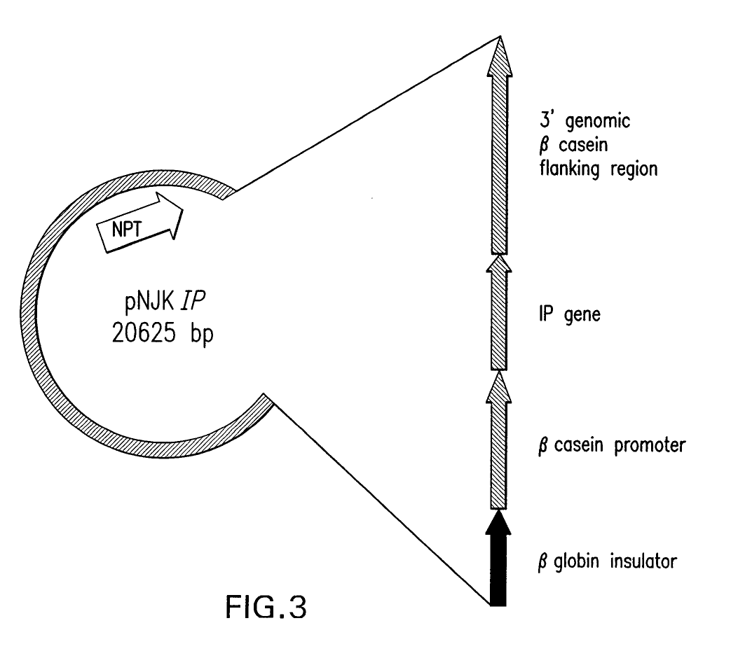

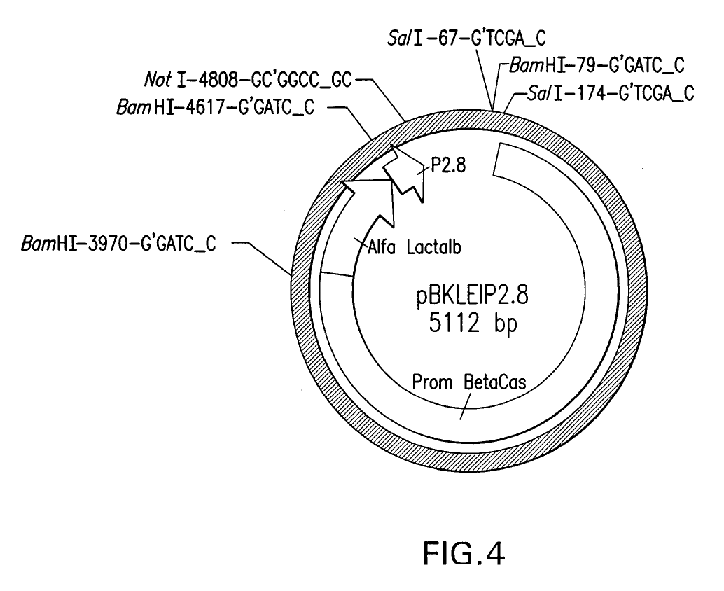

The invention also relates to a plasmid comprising a nucleic acid sequence encoding a modified insulin precursor operably linked to a beta casein promoter and an antibiotic resistance gene that allows for the selection of antibiotic resistant cells. Suitable beta casein promoters include, but are not limited to, a bovine beta casein promoter or a caprine beta casein promoter. Other beta casein promoters include, but are not limited to, a porcine beta casein promoter, an ovine beta casein promoter, or a rodent beta casein promoter. In embodiments, the antibiotic resistance gene is a neomycin resistance gene that allows for the selection of geneticin resistant cells. An example of such a plasmid is pβmhuIP, as shown in FIG. 1. Additional examples of such a plasmid are pNJK IP, as shown in FIG. 3, and pβKLE IP, as shown in FIG. 4.

The invention further relates to a plasmid comprising a nucleic acid sequence encoding a modified insulin precursor in which the modified insulin precursor is a modified mammalian insulin precursor. The modified mammalian insulin precursor may be a modified human insulin precursor, a modified bovine insulin precursor, a modified porcine insulin precursor, a modified ovine insulin precursor, a modified caprine insulin precursor, or a modified rodent insulin precursor. In embodiments, the modified mammalian insulin precursor is a modified human insulin precursor.

The invention further relates to a plasmid comprising a nucleic acid sequence encoding a modified insulin precursor that does not cause hypoglycemia in a non-human transgenic mammal that expresses the modified insulin precursor.

The invention further relates to a plasmid comprising a nucleic acid sequence encoding a modified human insulin precursor that contains a modified C peptide. In the modified human insulin precursor, the amino acids encoding the connecting C peptide that is found in naturally occurring human proinsulin is replaced by amino acids that are not found in naturally occurring proinsulin. In embodiments, the modified C peptide contains the following three amino acids: Ala-Ala-Lys. Furthermore, the modified human insulin precursor may contain a modified B chain. In embodiments, the modified B chain contains amino acids 1-29 of the naturally occurring B chain.

The invention further relates to a plasmid comprising a nucleic acid sequence encoding a modified insulin precursor, which further comprises one or more additional genetic elements that will enhance the stability of the plasmid, enhance the stability of the mRNA transcribed from the plasmid, decrease degradation of the modified insulin precursor, and/or increase the expression of the modified insulin precursor. Suitable genetic elements include, but are not limited to, a regulatory element (e.g., a promoter, an enhancer, an insulator, or a transcription termination site), a fragment of the coding sequence of a gene that is not insulin, or the coding sequence of a gene that is not insulin. In embodiments, the genetic element is a fragment of the coding sequence of the chicken β globin insulator. An example of such a plasmid is pNJK IP, as shown in FIG. 3. In other embodiments, the genetic element is a fragment of the coding sequence of the bovine alfa lactalbumin gene. An example of such a plasmid is pβKLE IP, as shown in FIG. 4.

The plasmids pβmhuIP, pNJK IP and pβKLE IP were deposited under the terms of the Budapest Treaty. The name and address of the depository are DSMZ—Deutsche Sammlung von Mikroorganismen und Zellkulturen GmbH, Inhoffenstr. 7B, D-38124 Braunschweig, Germany. pβmhuIP was deposited at the DSMZ on Apr. 4, 2008 and given DSMZ Deposit Number DSM 21359. pNJK IP was deposited at the DSMZ on Apr. 4, 2008 and given DSMZ Deposit Number DSM 21360. pβKLE IP was deposited at the DSMZ on Jun. 12, 2008 and given DSMZ Deposit Number DSM 21581

The invention further relates to a method of transfecting the above described genetic constructs. In embodiments, the above described genetic constructs are transfected into mammalian cells by inserting the genetic constructs into liposomes and contacting the liposomes with the mammalian cells. The liposomes may be cationic lipids.

Methods of selection of neomycin resistant cells in appropriate media are known to those of skill in the art. Such cells must be picked carefully, so as to avoid cell damage.

The invention also relates to a method of nuclear transfer of cells arrested in G₀, or at different times of the cell cycle, into enucleated mammalian oocytes, most preferably bovine oocytes.

The invention relates to a method of transgenic embryo transfer into the hormone stimulated uterus of a mammal, most preferably the uterus of a cow.

The invention further relates to a method of making a non-human transgenic mammal comprising cloning a nucleic acid sequence encoding the inactive protein of interest into a plasmid whereby the sequence is operably linked to a promoter that will direct the expression of the sequence in mammary cells, resulting in an expression plasmid; transfecting somatic cells with the expression plasmid so that the plasmid is incorporated into the genome of the cells, resulting in transgenic somatic cells; enucleating a mature oocyte, resulting in an enucleated oocyte; fusing one transgenic somatic cell with the enucleated oocyte resulting in a monocell embryo; implanting the embryo in the uterus of a receptive mammal; and monitoring the pregnancy through the birth of the transgenic mammal. The inactive protein of interest may be a modified insulin precursor that does not cause hypoglycemia in the mammal. The modified insulin precursor is preferably a modified mammalian insulin precursor, more preferably, a modified human insulin precursor, a modified bovine insulin precursor, a modified porcine insulin precursor, a modified ovine insulin precursor, a modified caprine insulin precursor, or a modified rodent insulin precursor, and, most preferably, a modified human insulin precursor. The non-human transgenic mammal may be, but is not limited to, a mammal of bovine species. Other species of transgenic mammals may be, but are not limited to, porcine species, ovine species, caprine species, or rodent species. The promoter may be a beta casein promoter. Suitable beta casein promoters include, but are not limited to, a bovine beta casein promoter or a caprine beta casein promoter. Other beta casein promoters include, but are not limited to, a porcine beta casein promoter, an ovine beta casein promoter, or a rodent beta casein promoter. The plasmid may also contain an antibiotic resistance gene such as the neomycin resistance gene. Further, the expression plasmid may be pβmhuIP. The expression plasmid may also be pNJK IP or pβKLE IP.

The invention further relates to a method of making a non-human transgenic mammal that expresses an inactive form of the protein of interest comprising a nucleic acid sequence encoding a modified insulin precursor that contains a modified C peptide. In the modified insulin precursor, the amino acids encoding the connecting C peptide that is found in naturally occurring proinsulin is replaced by amino acids that are not found in naturally occurring proinsulin. In embodiments, the modified C peptide contains the following three amino acids: Ala-Ala-Lys. Furthermore, the modified insulin precursor may contain a modified B chain. In embodiments, the modified B chain contains all but the C-terminal amino acid of the naturally occurring B chain.

The invention further relates to a method of making a non-human transgenic mammal that expresses an inactive form of the protein of interest in which the somatic cells may be fibroblasts. Additionally, the transgenic somatic cells may be isolated from a female transgenic that expresses an inactive form of the protein of interest in its milk. The transgenic somatic cells may be fibroblasts.

The invention further relates to a method of making a non-human transgenic mammal that expresses an inactive form of the protein of interest in which the nucleic acid sequence encoding the inactive form of the protein of interest is found in both the somatic cells and the germ cells of the mammal

The invention further relates to a method of making a non-human transgenic mammal of bovine species that produces a recombinant modified human insulin precursor in its milk, whose genome comprises an integrated plasmid, the plasmid comprising a nucleic acid sequence encoding the modified human insulin precursor and a beta casein promoter that directs expression of the sequence in mammary cells of the mammal. Suitable beta casein promoters are described above. The integrated plasmid may contain an antibiotic resistance gene such as the neomycin resistance gene. Further, the integrated plasmid may be pβmhuIP. The integrated plasmid may also be pNJK IP or pβKLE IP.

The invention further relates to a method of producing an inactive form of a protein of interest comprising making a non-human transgenic mammal which produces the inactive form of the protein of interest in its milk; obtaining the milk from the non-human transgenic mammal; purifying the inactive protein from the milk; converting the inactive form of the protein of interest in vitro; and purifying the protein of interest, wherein the protein of interest may be toxic to the non-human transgenic mammal. The inactive protein may be, but is not limited to, precursors, modified precursors or modified forms of the following: antibodies, hormones, growth factors, enzymes, clotting factors, apolipoproteins, receptors, drugs, pharmaceuticals, bioceuticals, nutraceuticals, oncogenes, tumor antigens, tumor suppressors, cytokines, viral antigens, parasitic antigens, and bacterial antigens. Preferably, the inactive protein may be a recombinant modified insulin precursor, more preferably, a recombinant modified mammalian insulin precursor, and most preferably, a recombinant modified human insulin precursor. The non-human transgenic mammal may be, but is not limited to, a mammal of bovine species. Other species of transgenic mammals may be, but are not limited to, porcine species, ovine species, caprine species, or rodent species.

The invention also relates to a method of producing an inactive form of a protein of interest in a non-human transgenic mammal that is made by a process that comprises cloning a nucleic acid sequence encoding the inactive form of the protein of interest into a plasmid whereby the sequence is operably linked to a promoter that will direct the expression of the sequence in mammary cells, resulting in an expression plasmid; transfecting somatic cells, optionally fibroblasts, with the plasmid so that the plasmid is incorporated into the genome of the somatic cells, resulting in transgenic somatic cells; enucleating a mature oocyte, resulting in an enucleated oocyte; fusing one transgenic somatic cell with the enucleated oocyte resulting in a monocell embryo; implanting the embryo in the uterus of a receptive mammal; and monitoring the pregnancy through the birth of the transgenic mammal. The inactive protein of interest may be a modified insulin precursor that does not cause hypoglycemia in the mammal. The modified insulin precursor is preferably a modified mammalian insulin precursor, more preferably, a modified human insulin precursor, a modified bovine insulin precursor, a modified porcine insulin precursor, a modified ovine insulin precursor, a modified caprine insulin precursor, or a modified rodent insulin precursor, and, most preferably, a modified human insulin precursor. The non-human transgenic mammal may be, but is not limited to, a mammal of bovine species. Other species of transgenic mammals may be, but are not limited to, porcine species, ovine species, caprine species, or rodent species. The promoter may be a beta casein promoter. Suitable beta casein promoters are described above. The plasmid can also contain an antibiotic resistance gene such as the neomycin resistance gene. Further, the expression plasmid may be pβmhuIP. The plasmid can also contain one or more additional genetic elements that will enhance the stability of the plasmid, enhance the stability of the mRNA transcribed from the plasmid, decrease degradation of the modified insulin precursor, and/or increase the expression of the modified insulin precursor. Suitable genetic elements include, but are not limited to, a regulatory element (e.g., a promoter, an enhancer, an insulator, or a transcription termination site), a fragment of the coding sequence of a gene that is not the protein of interest, or the coding sequence of a gene that is not the protein of interest. The expression plasmid can be pNJK IP or pβKLE IP.

In embodiments, the non-human transgenic mammal that expresses an inactive form of the protein of interest is cloned using a nucleic acid sequence encoding a modified insulin precursor that contains a modified C peptide. In the modified insulin precursor, the amino acids encoding the connecting C peptide that is found in naturally occurring proinsulin is replaced by amino acids that are not found in naturally occurring proinsulin. In embodiments, the modified C peptide contains the following three amino acids: Ala-Ala-Lys. Furthermore, the modified insulin precursor may contain a modified B chain. In embodiments, the modified B chain contains all but the C-terminal amino acid of the naturally occurring B chain.

In further embodiments, the non-human transgenic mammal that expresses an inactive form of the protein of interest is made by a process in which the somatic cells may be fibroblasts. Additionally, the transgenic somatic cells may be isolated from a female transgenic that expresses an inactive form of the protein of interest in its milk. The transgenic somatic cells may be fibroblasts.

In further embodiments, the nucleic acid sequence encoding the inactive form of the protein of interest is found in both the somatic cells and the germ cells of the non-human transgenic mammal that expresses the inactive form of the protein of interest.

The invention further relates to a method of producing an inactive form of human insulin in a non-human transgenic mammal that produces a recombinant modified human insulin precursor in its milk, whose genome comprises an integrated plasmid, the plasmid comprising a nucleic acid sequence encoding the modified human insulin precursor and a beta casein promoter that directs expression of the sequence in mammary cells of the mammal. Suitable beta casein promoters are described above. The integrated plasmid may contain an antibiotic resistance gene such as the neomycin resistance gene. Further, the integrated plasmid may be pβmhuIP. The integrated plasmid can also contain one or more additional genetic elements that will enhance the stability of the plasmid, enhance the stability of the mRNA transcribed from the plasmid, decrease degradation of the modified insulin precursor, and/or increase the expression of the modified insulin precursor. Suitable genetic elements include, but are not limited to, a regulatory element (e.g., a promoter, an enhancer, an insulator, or a transcription termination site), a fragment of the coding sequence of a gene that is not insulin, or the coding sequence of a gene that is not insulin. The integrated plasmid may be pNJK IP or pβKLE IP.

Additionally, the invention relates to a method of purifying an inactive form of the protein of interest from the milk of a transgenic mammal that produces the inactive protein in its milk. The purification method can include chromatography and filtration steps. Different types of chromatography can be employed, and include ion exchange chromatography or reverse phase chromatography. The ion exchange chromatography can be cation exchange chromatography. Further, multiple chromatography steps may be performed.

The invention further relates to a method of purifying an inactive form of a protein of interest from milk of a non-human transgenic mammal that produces the inactive protein in its milk, comprising obtaining the milk from the non-human transgenic mammal, clarifying the milk of the non-human transgenic mammal, resulting in clarified milk, and subjecting the clarified milk to chromatography, resulting in pure inactive protein. The chromatography steps may include ion exchange chromatography or reverse phase chromatography. The ion exchange chromatography may be cation exchange chromatography. The reverse phase chromatography may use reverse phase matrix such as C4 or C18 reverse phase matrixes. Further, multiple chromatography steps may be performed.

The invention further relates to a method of purifying an inactive form of a protein of interest from milk of a non-human transgenic mammal which produces the inactive protein in its milk, comprising obtaining the milk from the non-human transgenic mammal, clarifying the milk of the non-human transgenic mammal, resulting in clarified milk, subjecting the clarified milk to cation exchange chromatography, resulting in a cation exchange chromatographed material, subjecting the cation exchange chromatographed material to reverse phase chromatography, resulting in pure inactive protein.

The inactive protein of interest may be a recombinant modified insulin precursor, preferably, a recombinant modified mammalian insulin precursor, more preferably, a recombinant modified human insulin precursor, a recombinant modified bovine insulin precursor, a recombinant modified porcine insulin precursor, a recombinant modified ovine insulin precursor, a recombinant modified caprine insulin precursor, or a recombinant modified rodent insulin precursor, and, most preferably, a recombinant modified human insulin precursor. Additionally, the modified insulin precursor does not cause hypoglycemia in the non-human transgenic mammal that expresses the modified insulin precursor. In the modified insulin precursor, the amino acids encoding the connecting C peptide that is found in naturally occurring proinsulin is replaced by amino acids that are not found in naturally occurring proinsulin. In embodiments, the modified C peptide contains the following three amino acids: Ala-Ala-Lys. Furthermore, the modified insulin precursor may contain a modified B chain. In embodiments, the modified B chain contains all but the C-terminal amino acid of the naturally occurring B chain. The non-human transgenic mammals can be, but are not limited to, mammals of bovine species. Other species of transgenic mammals may be, but are not limited to, porcine species, ovine species, caprine species or rodent species.

The invention further relates to a method of converting an inactive form of the protein of interest into the mature (i.e., active) form of the protein of interest, and then purifying the protein of interest. The conversion can include enzymatic cleavage of the precursor of the protein of interest. The enzymatic cleavage can involve trypsinolysis. The purification of the protein of interest can include chromatography steps. These chromatography steps may include reverse phase chromatography. The reverse phase chromatography may use reverse phase matrix such as C4 or C18 reverse phase matrixes. Further, multiple chromatography steps may be performed.

The invention further relates to a method of converting a recombinant modified insulin precursor into recombinant insulin, and then purifying the recombinant insulin. The conversion can include enzymatic cleavage and transpeptidation of the recombinant modified insulin precursor. The enzymatic cleavage can involve trypsinolysis. The purification of the recombinant insulin can include chromatography steps. These chromatography steps may include reverse phase chromatography or ion exchange chromatography. Further, multiple chromatography steps may be performed.

The invention also relates to a method of converting a recombinant modified insulin precursor into recombinant insulin, and then purifying the recombinant insulin. This method comprises subjecting the recombinant modified insulin precursor to trypsinolysis and transpeptidation, resulting in a trypsinized and transpeptidated material, subjecting the trypsinized and transpeptidated material to a first reverse phase chromatography, resulting in a first reverse phase chromatographed material, subjecting the first reverse phase chromatographed material to a second reverse phase chromatography, resulting in a second reverse phase chromatographed material, and subjecting the second reverse phase chromatographed material to a third reverse phase chromatography, resulting in pure recombinant insulin. The steps of reverse phase chromatography include the use of reverse phase matrixes, preferably C4 or C18 reverse phase matrixes.

The recombinant insulin and the recombinant modified insulin precursor may be, respectively, a recombinant mammalian insulin and a recombinant modified mammalian insulin precursor, more preferably, recombinant human insulin and a recombinant modified human insulin precursor, recombinant bovine insulin and a recombinant modified bovine insulin precursor, recombinant porcine insulin and a recombinant modified porcine insulin precursor, recombinant ovine insulin and a recombinant modified ovine insulin precursor, recombinant caprine insulin and a recombinant modified caprine insulin precursor, or recombinant rodent insulin and a recombinant modified rodent insulin precursor, respectively, and, most preferably, recombinant human insulin and a recombinant modified human insulin precursor. Additionally, the modified insulin precursor does not cause hypoglycemia in the non-human transgenic mammal that expresses the modified insulin precursor. In the modified insulin precursor, the amino acids encoding the connecting C peptide that is found in naturally occurring proinsulin is replaced by amino acids that are not found in naturally occurring proinsulin. In embodiments, the modified C peptide contains the following three amino acids: Ala-Ala-Lys. Furthermore, the modified insulin precursor may contain a modified B chain. In embodiments, the modified B chain contains all but the C-terminal amino acid of the naturally occurring B chain.

The invention further relates to a method of producing a protein of interest comprising making a non-human transgenic mammal that produces an inactive form of the protein of interest in its milk, obtaining the milk from the non-human transgenic mammal, purifying the inactive from milk, in vitro converting the inactive protein by subjecting the purified inactive protein to enzymatic cleavage, and finally purifying the protein of interest.

The invention further relates to a method of producing recombinant insulin comprising making a non-human transgenic mammal that produces a recombinant modified insulin precursor in its milk, obtaining the milk from the non-human transgenic mammal, purifying the recombinant modified insulin precursor from milk, in vitro converting the precursor into recombinant insulin by subjecting the purified precursor to enzymatic cleavage and transpeptidation, and finally purifying the recombinant insulin.

Furthermore, the invention also relates to a method of producing recombinant insulin, comprising making a non-human transgenic mammal which produces a recombinant modified insulin precursor in its milk, obtaining the milk from the non-human transgenic mammal, clarifying the milk, resulting in clarified milk, subjecting the clarified milk to cation exchange chromatography, resulting in a cation exchange chromatographed material, subjecting the cation exchange chromatographed material to reverse phase chromatography, resulting in pure recombinant modified insulin precursor, subjecting the pure recombinant modified insulin precursor to trypsinolysis and transpeptidation, resulting in a trypsinized and transpeptidated material, subjecting the trypsinized and transpeptidated material to a first reverse phase chromatography, resulting in a first reverse phase chromatographed material, subjecting the first reverse phase chromatographed material to a second reverse phase chromatography, resulting in a second reverse phase chromatographed material, and subjecting the second reverse phase chromatographed material to a third reverse phase chromatography, resulting in pure recombinant insulin.

The recombinant insulin and the recombinant modified insulin precursor may be, respectively, a recombinant mammalian insulin and a recombinant modified mammalian insulin precursor, more preferably, recombinant human insulin and a recombinant modified human insulin precursor, recombinant bovine insulin and a recombinant modified bovine insulin precursor, recombinant porcine insulin and a recombinant modified porcine insulin precursor, recombinant ovine insulin and a recombinant modified ovine insulin precursor, recombinant caprine insulin and a recombinant modified caprine insulin precursor, or recombinant rodent insulin and a recombinant modified rodent insulin precursor, respectively, and, most preferably, recombinant human insulin and a recombinant modified human insulin precursor. The non-human transgenic mammal may be, but is not limited to, a mammal of bovine species. Other species of transgenic mammals may be, but are not limited to, porcine species, ovine species, caprine species or rodent species. The steps of reverse phase chromatography include the use of reverse phase matrixes, preferably C4 or C18 reverse phase matrixes.

The following examples are illustrative, but not limiting, of the various aspects and features of the present invention.

### Example 1

### Construction of the Expression Plasmid

A construct was generated that contained a large portion of the bovine beta casein gene promoter, including a short fragment of the 5′ non-coding beta casein gene region, fused to the coding sequence of a modified human insulin precursor. The short non-translated fragment is a fragment of the first exon of the beta casein gene. The beta casein region employed was about 3.8 kb.

The construction of the expression plasmid pβmhuIP (see FIG. 1) was carried out by inserting the coding sequence of the modified human insulin precursor (mhuIP) and a large portion of the bovine beta casein promoter gene (corresponding to 3,800 bp from the 5′ region of the beta casein bovine gene) into an adequate vector. This promoter ensures the tissue specific and developmentally regulated expression of genes under its control, in this case heterologous modified human insulin precursor.

For a proper selection of transgenic cells, a gene encoding Neomycin Phosphotransferase was included in the plasmid. This gene allows for the selection of transgenic cells with the antibiotic Geneticin, and it is under the control of the SV40 promoter.

Other constructs can be derived from the original one to improve transfected cell selection or DNA integration efficiency into the bovine cell genome.

Constructs were analyzed by restriction enzyme analysis and DNA sequencing. The ability of the constructs to express mhuIP was previously tested in a mammary gland cell line by fluorescent antibody recognition.

The preparation of the plasmid pβmhuIP is described below in detail.

Preparation of pβmhuIP

A Start Construction was generated, which includes the sequence encoding mhuIP (human proinsulin containing a modified C peptide). mhuIP is similar to human proinsulin except that the C peptide in mhuIP is shorter than the C peptide found in naturally occurring proinsulin.

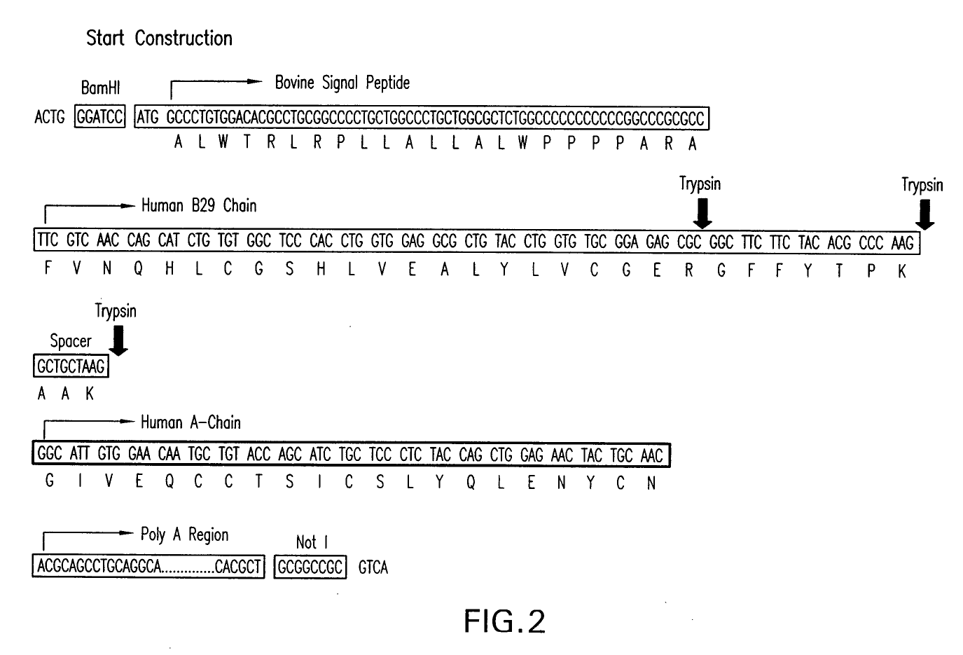

FIG. 2 depicts a scheme of the Start Construction. At the beginning, a region encoding a bovine signal peptide can be found, followed by the sequence encoding the B Chain of insulin (lacking the C-terminal amino acid). Then, a region encoding a spacer of three amino acids, Ala-Ala-Lys, can be found, which is followed by the sequence encoding the full A Chain of insulin, and finally a region encoding the mRNA poly A. The three amino acid spacer, Ala-Ala-Lys, replaces the C peptide found in naturally occurring proinsulin.

The Start Construction was generated by rebuilding the mhuIP sequence from 6 overlapping, chemically synthesized oligonucleotides containing the recognition sites for restriction enzymes Bam HI and Not I. The primers sequences are shown below:

Ins1:
5′-ACTGGGATCCATGGCCCTGTGGACACGCCTGCGGCCCCTGCTGGCCC
TGCTGGCGCTCTGGCCCCCCCCCCCGGCCCG-3′
Ins2:
5′-CTCCGCACACCAGGTACAGCGCCTCCACCAGGTGGGAGCCACACAGA
TGCTGGTTG ACGAAGGCGCGGGCCGGGGGGGGG-3′
Ins3:
5′-ACCTGGTGTGCGGAGAGCGCGGCTTCTTCTACACGCCCAAGGCTGCT
AAGGGCATT GTGGAACAATGCTGTACCAG-3′
Ins4:
5′-GTGTGGGGCTGCCTGCAGGCTGCGTCTAGTTGCAGTAGTTCTCCAGC
TGGTAGAGG GAGCAGATGCTGGTACAGCA-3′
Ins5:
5′-CAGGCAGCCCCACACCCGCCGCCTCCTGCACCGAGAGAGATGGAATA
AAGCCCTTG AACCAGCCCTGCTGTGCCGTCTGT-3′
Ins6:
5′-TGACGCGGCCGCAGCGTGGAGAGAGCTGGGAGGGGCTCACAACAGTG
CCGGGAAG TGGGGCTTGGCCCAGGGCCCCCAAGACACACAGCAGGCACA
GCA-3′

The rebuilding process was performed by PCR. PCR products were generated from mixes of primers Ins1 and Ins2 (product f12), Ins3 and Ins4 (product f34) and Ins5 and Ins6 (product f56). The same process was then performed using f12 and f34 overlapping fragments in a single mix, which renders the product f14. Finally, the f14 product was used in a PCR in a mix containing f56 to amplify a fragment of approximately 410 bp, comprising the full length mhuIP (fragment f16).

Once the fragment f16 was obtained, it was cloned into an adequate vector and transformed into competent E. coli bacterial cells for further amplification of the cloning vector with its corresponding insert. The vector was derived from pBKCMV. pBKCMV is an expression vector available from Invitrogen Co. (Carlsbad, Calif.), which encodes a CMV promoter, a neomycin resistance gene, and a kanamycin resistance gene. The CMV promoter was replaced with a 3.8 kb bovine beta casein promoter and fragment f16 was cloned into the resulting vector using the Bam HI and Not I restriction sites.

After amplification, restriction tests were performed in order to check the identity of the cloned insert. Final confirmation was achieved by sequencing.

Afterwards, the Start Construction was directionally inserted (Bam HI/Not I) in a plasmid vector downstream to a bovine beta casein promoter of 4 kB. The plasmid vector also contained a neomycin resistance gene. The resulting vector, pβmhuIP, which is the final construct, contained the beta casein promoter, the sequence encoding mhuIP, and the neomycin resistance gene.

Human proinsulin is made up of three domains: an amino-terminal B chain (30 amino acids), a carboxyl-terminal A chain (21 amino acids), and a connecting peptide in the middle known as the C peptide (31 amino acids). mhuIP differs from the naturally occurring form of human proinsulin in that the C-terminal amino acid of the B chain has been removed and the amino acids encoding the C peptide have been replaced with three amino acids that are normally not found in the C peptide, Ala-Ala-Lys. Mature human insulin, which is made up of only the A chain and the B chain, is formed after cleavage of the C peptide. A transgenic mammal expressing human proinsulin is nonviable because host peptidases can cleave and remove the C peptide, forming mature insulin. As described above, expression of mature human insulin in a non-human transgenic mammal may kill the mammal because the mature human insulin may leak into the blood stream of the mammal. In contrast, a non-human transgenic mammal made using pβmhuIP expresses a modified human insulin precursor that will not cause the transgenic mammal to develop any significant hypoglycemia and will not be toxic to the transgenic mammal. Without being limited to the following, because the region encoding the three amino acid spacer of the modified human insulin precursor differs from the C peptide found in naturally occurring human proinsulin, host peptidases do not recognize and cleave the three amino acid spacer. Consequently, the modified human insulin precursor remains inactive and does not cause hypoglycemia in the transgenic animal, which is an important advantage of the claimed invention.

Clones were selected which contain beta casein promoter and mhuIP properly fused to express mhuIP only under the control of this promoter.

The size of this expression plasmid is about 8.4 kbp.

### Transfection of Somatic Cells

The plasmid pβmhuIP was then used for transfecting a primary culture of somatic cells, using calcium phosphate or a liposome method. Fetal calf fibroblasts were generally employed for the transfection.

The transfected cells were selected by adding Geneticin to the culture. After a period of 2 to 8 weeks, the cells that were resistant to Geneticin were suitable for being used as donor cells to obtain transgenic clones. Transfected selected cells were analyzed by PCR to verify that the cells contained the expression cassette.

### Example 2

### Oocyte Enucleation and Metaphase Nuclear Transfer in Mature Enucleated Oocytes

### Collection and In Vitro Maturation of Bovine Oocytes

Bovine oocytes were aspirated from slaughterhouse ovaries and matured in TCM-199+5% FCS+3 mM HEPES+antimycotics. The selected oocytes were then placed in TCM-199+Roscovitine, under atmosphere of 5% CO₂ at 39° C. for 20 hs. Afterwards, oocytes were placed in TCM-199+5% FCS+FSH (follicle-stimulating hormone)+antibiotics under atmosphere of 5% CO₂ at 39° C. for 24 hs. Mature oocytes were denuded by vortexing for 2 minutes in PBS with 1 mg/ml bovine testis hyaluronidase.

### Example 3

### Nuclear Transfer with Cumulus Cells

### Enucleation

Oocytes were mechanically enucleated using a Narishige hydraulic micromanipulators and Nikon Diaphot microscopy. Enucleation was performed with 20 μm beveled and sharpened pipettes. Oocytes were previously stained with 5 μg/ml bisbenzimidine (Hoechst 33342¹) dye for 20 minutes. Metaphases were enucleated by visualization of the stained chromosomes under ultraviolet light. Metaphase chromosomes were assessed after aspiration inside the pipette. A transgenic somatic cell was transferred into the perivitelline space and tightly opposed to the enucleated oocyte. ¹ Sigma Chemical Co., St. Louis, Mo., USA.

### Fusion, Activation and Embryo Culture

A transgenic somatic cell and an enucleated oocyte were manually aligned in the fusion chamber so that the membranes to be fused were parallel to the electrodes. This was done using a glass embryo-handling pipette.

Fusion was performed using one electrical pulse of 180 volts/cm for 15 μs (BTX Electro Cell Manipulator 200)² and monitored with a BTX Optimizer-Graphic Pulse Analyzer. The chamber for pulsing embryos consisted of two 0.5 mm stainless steel wire electrodes mounted 0.5 mm apart on glass microscope slide. Three hours after fusion, activation was induced by incubation in TL-HEPES with 5 μM ionomycin for 4 min and in TCM-199 with 2 mM 6-DMAP for 3 hours. ² BTX Inc., San Diego, Calif., USA.

The activated oocytes were then cultured in SOF medium under atmosphere of 5% CO₂+5% O₂+90% N₂ for 6.5 days, until development of blastocysts.

Afterwards, embryo transfer into surrogate cows took place. Generally, two blastocysts per recipient cow were non-surgically transferred, and pregnancies at 30-35 days were determined by ultrasonography.

The implanted cows were allowed to normally pass the pregnancy up to a natural delivery. Eventually a chirurgic approach (Caesarea) could be used for delivery. The newborns were fed with Ig rich colostrum during the first 48 hours, and then synthetic, later natural (all of them free of animal origin compounds) foods were used.

### Example 4

### Tests Performed on Transgenic Calves

In the current example, we present a full description of the tests performed on a particular transgenic calf, which was obtained as a result of the procedure described in Examples 1 to 3. Nonetheless, it should remain clear that the same set of assays could be performed on bovine that are born as a consequence of other methods for obtaining transgenic calves, such as subcloning of transgenic females, superovulation of a transgenic female followed by artificial insemination, or artificial insemination of transgenic or non-transgenic females with semen from a bull which is transgenic for the desired protein.

It was proved by means of PCR reactions performed on DNA purified from the calf's white blood cells, using DNA from non-transgenic jersey calves as the negative control, that bovine beta casein promoter and the sequence encoding modified human insulin precursor are included in the transgenic calf cells genome. They can be found together as a unique DNA fragment that is different from the homologue beta casein gene of the calf.

It was corroborated, by using a Pharmacia automatic sequencer, that the inserted sequence corresponds to the sequence encoding the modified human insulin precursor contained in the cloning plasmid. The inserted sequence includes the secretion signal and terminator. The bovine beta casein promoter that controls the expression of the modified human insulin precursor sequence in our calf was sequenced, too. All those elements coincide exactly with the expected theoretical sequence from the genetic construct used to transform the cells out of which the clones were generated.

### Example 5

### Purification of Recombinant mhuIP from Milk, Conversion of mhuIP into Human Insulin and Purification of Human Insulin

An amount of recombinant mhuIP protein, necessary for the development of the purification procedure of the precursor from milk, was obtained from fermentation of transformed Pichia pastoris. For this purpose, the sequence encoding mhuIP was subcloned in an expression vector downstream a yeast secretion signal sequence, under the control of a promotor inducible by methanol, and transformed in yeast cells.

After that, selection of a proper clone was performed and a liquid culture was made from the selected clone.

Fermentation of the transformed yeast clone was made in a medium containing glycerol as the carbon source, oligoelements. Methanol was used for induction. This fermentation rendered 0.5 grams of mhuIP per liter of culture.

Once the fermentation was over, purification steps were carried out to achieve a pure product.

The initial goal was to obtain pure recombinant mhuIP from the yeast culture. Thus, the supernatant of a transformed Pichia pastoris culture was diluted tenfold with purified water and its pH was adjusted to 3.0 using Glacial Acetic Acid. The conductivity of this solution was verified so that it did not exhibit a value higher than 7 mS/cm. A purification process was afterwards performed, in order to obtain the starting material for the development of the purification process of the recombinant mhuIP from milk of transgenic mammals, and its ulterior conversion into recombinant human insulin.

First, the aforementioned solution was subjected to a cationic exchange chromatography step employing SP Sepharose FF resin (Amersham), at a flow rate of 100 cm/h. Loading (10 mL of supernatant were loaded per mL of resin) and equilibration of the column were performed employing 5% Acetic acid. For the elution of the protein a gradient of 5% Acetic acid:1M NaCl at pH 3 was applied, starting from a 100:0 ratio of the solutions until a 0:100 ratio of the solutions in a total volume of 25 volumes of the column was reached.

In a second purification step, the eluate from the cationic exchange chromatography was subjected to reverse phase chromatography. The previous eluate was diluted fivefold with purified water, and the pH was adjusted to 3 with trifluoroacetic acid (TFA).

The resulting solution was loaded into a column containing C4 Baker Wide Pore resin. The flow was set at a rate of 100 cm/h. For loading and equilibration, 0.1% TFA/water was used. For elution, a gradient 0.1% TFA/water-Acetonitrile was applied, starting from a 100:0 ratio of the solutions until a 0:100 ratio in a total volume of 50 volumes of the column was reached.

The overall yield of the previous purification steps was approximately 42% and mhuIP with a purity degree higher than 95% was obtained.

A process comprising the purification of the recombinant mhuIP from milk (since the transgenic mammals will secrete the precursor in their milk), the conversion of the recombinant mhuIP into recombinant human insulin and the final purification of recombinant human insulin was developed. The starting material for this development was obtained by mixing the pure recombinant mhuIP (obtained from Pichia pastoris, as described above) with regular cow milk.

An exhaustive purification process was developed. This process comprised the steps of: obtaining the skim of the milk by means of tangential filtration and dilution of the obtained eluate to achieve a better solubility of recombinant mhuIP eventually retained in the micelles of casein (clarification); and passage of this solution through a cationic exchange chromatography column. The resulting solution was subjected to a reverse phase chromatography (C4) step and fractions rich in recombinant mhuIP were afterwards subjected to trypsinolysis and transpeptidation. Finally, purification of the recombinant human insulin took place, since it is mandatory, when manufacturing a biopharmaceutical product, that the protein of interest should be purified to homogeneity in order to avoid the presence of possible contaminants in the product.

The procedure for the purification of the recombinant mhuIP from milk, the later conversion into recombinant human insulin and the final purification of recombinant human insulin, comprises the following steps in order: (a) tangential flow filtration (clarification), (b) cationic exchange chromatography, (c) reverse phase chromatography (C4), (d) trypsinolysis and transpeptidation, (e) reverse phase chromatography (C4), (f) reverse phase chromatography (C4) and (g) reverse phase chromatography (C18).

### Clarification

Fresh milk was mixed with a sufficient amount of pure recombinant mhuIP, produced in P. pastoris as described previously. Afterwards, the product was subjected to a tangential flow filtration step. Filter pore size was 0.1 μm and the process yield was 80%.

### Cationic Exchange Chromatography

The material resulting from the previous step was chromatographed employing a cationic exchange matrix. The pH of the solution to be chromatographed was adjusted to 3.0 with Glacial Acetic Acid. The conductivity was checked so that it was not higher than 7 mS/cm.

This chromatography step was performed employing SP Sepharose FF resin (Amersham) at a flow rate of 100 cm/h. Loading and equilibration of the column was performed employing 5% Acetic acid. For the elution of the protein a gradient of 5% Acetic acid-1M NaCl at pH 3 was applied, starting from a 100:0 ratio of the solutions until a 0:100 ratio of the solutions in a total volume of 25 volumes of the column was reached.

The chromatography step had a yield of 90%. The selected recombinant mhuIP containing fractions were assayed for total proteins (by Bradford method) and for the protein of interest (by Western Blot), and stored at 2-8° C.

### Reverse Phase Chromatography (1)

The material resulting from the previous step was then subjected to reverse phase chromatography employing C4 Baker Wide Pore resin. The flow was set at a rate of 100 cm/h. For loading and equilibration, 0.1% TFA/water was used. For elution, a gradient of 0.1% TFA/water-Acetonitrile was applied, starting from a 100:0 ratio of the solutions until a 0:100 ratio of the solutions in a total volume of 50 volumes of the column was reached.

This step had a yield of 68%.

### Trypsinolysis and Transpeptidation

The material resulting from the previous step was treated with trypsin.

For trypsinolysis, a 10 mM mhuIP solution was incubated with Trypsin (in a concentration of 200 μM) at 12° C. for 24 hours

Once the incubation was finished, the transpeptidation reaction was performed in order to add the Threonine in position 30 of the B Chain of human insulin. For this purpose, a solution containing 0.8 M Thr-Obu, 50% DMF/EtOH (1:1), 26% H2O, Acetic acid, 10 mM mhuIP, and 200 μM Trypsin was prepared, and the transpeptidation reaction was allowed to progress until completion.

Once the transpeptidation step had finished, the resulting solution was subjected to three successive reverse phase chromatography steps in order to yield pure recombinant human insulin.

### Reverse Phase Chromatography (2)

The material resulting from the previous step was subjected to reverse phase chromatography.

First, a dilution of the material of the previous digestion was up to 0.125 mg/mL using 50 mM NaH₂PO₄, pH 5.0. Afterwards, 50 μL of Acetic acid per 100 mL of solution was added. The sample was then clear, the pH was around 4.5, and the sample was ready to be loaded.

Buffers compositions are described below:

Mobile Phase A (MPA): 210 mL sulfate buffer+790 mL of purified water (conductivity around 48 mS)

Mobile Phase B (MPB): 105 mL sulfate buffer+40% Acetonitrile, purified water q.s.p. to 1 L.

1 L of sulfate buffer contains 132.1 gr. of NH₄SO₄, 14 mL of H₂SO₄ and its pH is adjusted at 2.00.

After loading, the elution was performed at a flow rate of 100 cm/h as follows: first, a gradient MPA-MPB was applied, starting from a 100:0 ratio of the solutions until a 55:45 ratio of the solutions in a total volume of 135 mL was reached; afterwards, another gradient MPA-MPB was applied, starting from a 55:45 ratio of the solutions until a 25:75 ratio of the solutions in a total volume of 360 mL was reached; and, last, a final gradient MPA-MPB was applied, starting from a 25:75 ratio of the solutions until a 0:100 ratio of the solutions in a total volume of 50 mL was reached.

The obtained fraction contains recombinant human insulin with a purity of over 98% and the yield of this step is around 85%.

### Reverse Phase Chromatography (3)

The material resulting from the previous step was conditioned for this step by adjusting its pH to 7.4.

The resulting solution was then chromatographed employing a C4 Baker Wide Pore matrix. The flow was set at a rate of 100 cm/h. For loading and equilibration, 0.1% TFA/water was used. For elution, a gradient of 0.1% TFA/water-Acetonitrile was applied, starting from a 100:0 ratio of the solutions until a 0:100 ratio of the solutions in a total volume of 50 volumes of the column was reached.

This step had a yield of approximately 65%.

### Reverse Phase Chromatography (4)

The material obtained in the previous step was conditioned for a final reverse phase chromatography step by adjusting its pH to 3.0.

The conditioned material was then chromatographed employing a C18 reverse phase matrix. The flow was set at a rate of 100 cm/h. For loading and equilibration, 0.1% TFA/water was used. For elution, a gradient of 0.1% TFA/water-Acetonitrile was applied, starting from a 100:0 ratio of the solutions until a 0:100 ratio of the solutions in a total volume of 50 volumes of the column was reached.

This step had a yield of approximately 61%.

### Example 6

### Construction of the Expression Plasmid pNJK IP

An alternative construct to express mhuIP in transgenic bovine mammary glands was generated, containing a large portion of the caprine beta casein gene promoter, fused to a fragment of the coding sequence of the chicken β globin insulator. Insulators are DNA sequence elements that shield a promoter from nearby regulatory elements, including nearby silencing sequences that inhibit gene expression. This alternative construct containing the chicken P globin insulator was generated in order to block inhibition of the beta casein promoter by any nearby silencing sequences.

The construction of this alternative plasmid was carried out, first, by excising from pBCl, which is a commercial vector available from Invitrogen Co. (Carlsbad, Calif.), a 15 kb fragment containing: a 2.4 kb fragment of the chicken β globin insulator, a 3.1 kb caprine beta casein promoter sequence, including the introns and the nontranslatable exons from the caprine beta casein gene, a Xho I cloning site between the introns and the nontranslatable exons from the caprine beta casein gene, and the poly A signal and the flanking regions from the 3′ beta casein genomic sequence.

This 15 kb fragment was cloned into the backbone of pβmhuIP. First, the 3.8 kb bovine beta casein promoter and the mhuIP fragment from pβmhuIP was excised. The 15 kb fragment was inserted into this vector using the Sal I and Not I restriction sites. Then, the 410 bp mhuIP f16 fragment was cloned into the Xho I cloning site located in the 15 kb fragment (between the introns and the nontranslatable exons from the caprine beta casein gene, as described above).

The resulting vector (pNJK IP, FIG. 3) was transformed into competent E. coli bacterial cells for further amplification of the cloning vector with its corresponding insert.

After amplification, restriction site analysis was performed to check the orientation of the mhuIP f16 fragment insert. Final confirmation was achieved by sequencing.

### Example 7

### Construction of the Expression Plasmid pβKLE IP

An alternative construct to express mhuIP in transgenic bovine mammary glands was generated, containing a large portion of the bovine beta casein gene promoter, including a short non-translated fragment of the first exon of the beta casein gene, fused to the coding sequence of a large portion of the bovine alfa lactalbumin gene, followed by an enterokinase cleavage site, which is followed by the coding sequence of the modified human insulin precursor (mhuIP). This alternative construct expresses an alfa lactalbumin-mhuIP fusion protein. Because of its larger sequence, this alternative construct should yield mRNA with higher stability. In addition, because alfa lactalbumin is a protein naturally expressed in the bovine mammary gland, this alternative construct should minimize mhuIP degradation and increase mhuIP expression

In order to generate this construct, first, a PCR reaction was performed using DNA from leucocytes of bovine peripheral blood as template, to clone a large portion of the alfa lactalbumin gene. The PCR product comprised about 700 bp of alfa lactalbumin up to the end of the second exon and included the alfa lactalbumin signal sequence.

The first PCR reaction employed the following oligonucleotides:

| | |
|---|---|
| NES: | GGA GGT GAG CAG TGT GGT GAC |
| ALB: | GAA GTT ACT CAC TGT CAC AGG AGA |

Then, a second PCR reaction was performed, employing the following oligonucleotides:

| | |
|---|---|
| SIG: | TCA CCA AAA TGA TGT CCT TTG TC |
| LAC: | TGT CAC AGG AGA TGT TAC AGA |

Through this procedure, a 620 bp fragment was obtained and cloned intp pUC using the Sma I restriction site (pUC alfa lactalbumin).

The mhuIP gene bearing fragment was obtained by PCR from an in-house IP cloning plasmid, with the following oligonucleotides:

| | |
|---|---|
| EKB: | TAG GCT AGC GAT GAT GAT GAT AAA TTC GTT AAC |
| | CAG CAC CTG |
| CadAr: | TCA GCG GCC GC TTA GTT GCA GTA GTT |

The resultant 260 bp fragment included a short sequence coding for the enterokinase recognition site upstream from the coding sequence of mhuIP. The enterokinase recognition site will separate the mhuIP and alfa lactalbumin genes in the final expression plasmid. The 260 bp fragment was then digested with NheI and inserted into compatible Xba I restriction sites in pUC alfa lactalbumin. A resultant construct was selected in which the 260 bp fragment was positioned downstream from the alfa lactalbumin gene in the correct orientation. This construct has the coding sequence of a large portion of the bovine alfa lactalbumin gene, including the alfa lactalbumin signal sequence, followed by an enterokinase recognition site, which is further followed by the coding sequence of the modified human insulin precursor (mhuIP).

This construct was then digested with EcoRI, and the resulting digest product was Klenow treated. The resultant blunt ended product was then digested with NotI, and the fragment of the digest product containing the alfa lactalbumin gene and the mhuIP coding sequence was purified. The purified digest product was then inserted into the beta casein promoter expression plasmid, pBKCMV. Specifically, pBKCMV was first digested with NotI. Second, pBKCMV was further digested with EcoRI, and the resulting pBKCMV digest product was Klenow treated to generate an EcoRI blunted end. Finally, the purified digest product containing the alfa lactalbumin gene and the mhuIP coding sequence was inserted into pBKCMV using the EcoRI blunted and the NotI digested ends. The resulting vector, pβKLE IP (FIG. 4), contains a bovine beta casein promoter, followed by a large portion of the bovine alfa lactalbumin gene, including the alfa lactalbumin signal sequence, followed by an enterokinase recognition site, which is further followed by the coding sequence of the modified human insulin precursor (mhuIP).

pβKLE IP was then transformed into competent E. coli bacterial cells for further amplification of the cloning vector with its corresponding insert.

After amplification. final confirmation was achieved by sequencing.

Having now fully described the invention, it will be understood by those of ordinary skill in the art that the same can be performed within a wide and equivalent range of conditions, formulations and other parameters without affecting the scope of the invention or any embodiment thereof. All patents and publications cited herein are fully incorporated by reference herein in their entirety.

1-23. (canceled)

24. A non-human transgenic mammal which produces a modified insulin precursor in its milk.

25. The non-human transgenic mammal of claim 24, wherein the mammal is of bovine species, porcine species, ovine species, caprine species or rodent species.

26. The non-human transgenic mammal of claim 25, wherein the mammal is of bovine species.

27. The non-human transgenic mammal of claim 24, wherein the modified insulin precursor is a modified mammalian insulin precursor.

28. The non-human transgenic mammal of claim 27, wherein the modified mammalian insulin precursor is a modified human insulin precursor, a modified bovine insulin precursor, a modified porcine insulin precursor, a modified ovine insulin precursor, a modified caprine insulin precursor or a modified rodent insulin precursor.

29. The non-human transgenic mammal of claim 28, wherein the modified mammalian insulin precursor is a modified human insulin precursor.

30. The non-human transgenic mammal of claim 24, wherein the modified insulin precursor does not cause hypoglycemia in the non-human transgenic animal.

31. The non-human transgenic mammal of claim 30, wherein the modified insulin precursor comprises a modified C peptide.

32. The non-human transgenic mammal of 31, wherein the modified C peptide comprises amino acids that are not normally found in naturally occurring proinsulin.

33. The non-human transgenic mammal of 32, wherein the modified C peptide comprises the following three amino acids: Ala-Ala-Lys.

34. The non-human transgenic mammal of claim 31, wherein the modified insulin precursor further comprises a modified B chain.

35. The non-human transgenic mammal of claim 34, wherein the modified B chain comprises all but the C-terminal of the naturally occurring B chain.

36. The non-human transgenic mammal of claim 24, whose genome comprises an integrated plasmid, wherein the plasmid comprises a sequence encoding a modified insulin precursor operably linked to a promoter that directs the expression of the sequence in mammary cells of the mammal.

37. The non-human transgenic mammal of claim 36, wherein the mammal is of bovine species, porcine species, ovine species, caprine species or rodent species.

38. The non-human transgenic mammal of claim 37, wherein the mammal is of bovine species.

39. The non-human transgenic mammal of claim 24, wherein the modified insulin precursor is a modified mammalian insulin precursor.

40. The non-human transgenic mammal of claim 39, wherein the modified mammalian insulin precursor is a modified human insulin precursor, a modified bovine insulin precursor, a modified porcine insulin precursor, a modified ovine insulin precursor, a modified caprine insulin precursor or a modified rodent insulin precursor.

41. The non-human transgenic mammal of claim 40, wherein the modified mammalian insulin precursor is a modified human insulin precursor.

42. The non-human transgenic mammal of claim 41, wherein the promoter is a beta casein promoter.

43. The non-human transgenic mammal of claim 42, wherein the plasmid further comprises an antibiotic resistance gene.

44. The non-human transgenic mammal of claim 43, wherein the antibiotic resistance gene is a neomycin resistance gene.

45. The non-human transgenic mammal of claim 44, wherein the plasmid is pβmhuIP.

46. A non-human transgenic mammal of bovine species which produces a modified human insulin precursor in its milk, whose genome comprises an integrated plasmid, wherein the plasmid comprises a sequence that encodes a modified human insulin precursor and a beta casein promoter that directs the expression of the sequence in mammary cells of the mammal.

47. The non-human transgenic mammal of claim 46, wherein the plasmid further comprises a neomycin resistance gene.

48. The non-human transgenic mammal of claim 47, wherein the plasmid is pβmhuIP.

49. The non-human transgenic mammal of claim 48, wherein the integrated plasmid is found in somatic cells and germ cells of the mammal.

50. The non-human transgenic mammal of claim 46, wherein the modified human insulin precursor does not cause hypoglycemia in the non-human transgenic animal.

51. The non-human transgenic mammal of claim 50, wherein the modified human insulin precursor comprises a modified C peptide.

52. The non-human transgenic mammal of claim 51, wherein the modified C peptide comprises amino acids that are not normally found in naturally occurring proinsulin.

53. The non-human transgenic mammal of claim 52, wherein the modified C peptide comprises the following three amino acids: Ala-Ala-Lys.

54. The non-human transgenic mammal of claim 51, wherein the modified human insulin precursor further comprises a modified B chain.

55. The plasmid of claim 54, wherein the modified B chain comprises all but the C-terminal of the naturally occurring B chain.

56-77. (canceled)
